(19) United States
(12) Patent Application Publication
(10) Pub. No.: US 2025/0302712 A1
(43) Pub. Date: Oct. 2, 2025

(54) COMPOSITION FOR SKIN MOISTURIZING COMPRISING PHENYLLACTIC ACID

(71) Applicant: AMOREPACIFIC CORPORATION, Seoul (KR)

(72) Inventors: Kilsun MYOUNG, Yongin-si (KR); Suyeon KIM, Yongin-si (KR); Shinhyoung KIM, Yongin-si (KR); Hyewon MOK, Yongin-si (KR); Donghyun CHO, Yongin-si (KR); Eun Jeong CHOI, Yongin-si (KR); Saitbyul PARK, Yongin-si (KR)

(73) Assignee: AMOREPACIFIC CORPORATION, Seoul (KR)

(21) Appl. No.: 19/063,901

(22) Filed: Feb. 26, 2025

(30) Foreign Application Priority Data
Mar. 26, 2024 (KR) 10-2024-0040971

(51) Int. Cl. A61K 8/365; A61K 8/99; A61Q 19/00

(52) U.S. Cl. CPC A61K 8/365; A61K 8/99; A61Q 19/007

(57) ABSTRACT

Disclosed herein are a method for moisturizing a skin and a method for producing phenyllactic acid. An effective amount of a composition comprising at least one selected from the group consisting of phenyllactic acid; a strain having phenyllactic acid production ability; a lysate thereof; a culture thereof; a fermentation thereof; and an extract of the strain, lysate, culture or fermentation thereof, according to an embodiment of the present disclosure, can effectively increase the expression of hyaluronic acid producing enzymes in skin cells, thereby protecting the skin from dryness caused by blue light, ultraviolet rays, etc., and therefore can be administered to a skin moisturizing cosmetic composition, a quasi-drug composition, a general food composition, or a health functional food composition.

### CROSS REFERENCE TO RELATED APPLICATION

The present application claims priority to Korean Patent Application No. 10-2024-0040971, filed Mar. 26, 2024, the entire contents of which are hereby incorporated by this reference.

### FIELD OF THE INVENTION

Disclosed herein are a method for moisturizing a skin and a method for producing phenyllactic acid.

### BACKGROUND OF THE INVENTION

Phenyllactic acid (PLA) is a metabolite derived from phenylalanine, and is transformed into phenyllactic acid by lactate dehydrogenase after the intermediate metabolite phenylpyruvic acid is generated. Phenyllactic acid can be observed in various fermented foods and lactic acid bacteria culture mediums. Representative effects of phenyllactic acid include growth inhibition against harmful bacteria such as Klebsiella pneumonia, Staphylococcus aureus, and Aspergillus flavus, and based on this antibacterial activity, phenyllactic acid is used as an additive for long-term preservation in foods or animal feed.

The skin-related effects of phenyllactic acid are known as a topical skin agent for improving wrinkles and that it can induce a skin-whitening effect by inhibiting the activity of tyrosinase, but its moisturizing effects are not known.

Meanwhile, hyaluronic acid is a type of glycosaminoglycan, a chain-shaped polymer polysaccharide substance in which glucuronic acid and N-acetylglucosamine residues are repeatedly linked. Hyaluronic acid is a major component of the extracellular matrix, and it has been reported to be involved in moisture retention, intercellular spacing, storage and diffusion of cell growth factors and nutrients, as well as cell division, differentiation, and movement. Therefore, increasing the amount of hyaluronic acid biosynthesis can help moisturize the skin.

### SUMMARY OF THE INVENTION

One object of the present disclosure is to provide a method for moisturizing a skin.

Another object of the present disclosure is to provide a method for producing phenyllactic acid.

In order to achieve the above object, one aspect of the present disclosure provides a method for moisturizing a skin or promoting hyaluronic acid production, comprising administering to a subject an effective amount of a composition comprising at least one selected from the group consisting of phenyllactic acid; a strain having phenyllactic acid production ability; a lysate thereof, a culture thereof, a fermentation thereof, and an extract of the strain, lysate, culture or fermentation thereof as an active ingredient.

In another aspect, the present disclosure provides a method for producing phenyllactic acid comprising culturing a Lactobacillus plantarum strain.

The composition comprising at least one selected from the group consisting of phenyllactic acid; a strain having phenyllactic acid production ability; a lysate thereof, a culture thereof; a fermentation thereof, and an extract of the strain, lysate, culture or fermentation thereof as an active ingredient, according to an embodiment of the present disclosure, can effectively increase the expression of hyaluronic acid producing enzymes in skin cells, thereby protecting the skin from dryness caused by blue light, ultraviolet rays, etc., and therefore can be administered to a skin moisturizing cosmetic composition, a quasi-drug composition, a general food composition, or a health functional food composition.

The composition comprising at least one selected from the group consisting of phenyllactic acid; a strain having phenyllactic acid production ability; a lysate thereof, a culture thereof; a fermentation thereof, and an extract of the strain, lysate, culture or fermentation thereof as an active ingredient, according to an embodiment of the present disclosure, can effectively increase the expression of hyaluronic acid producing enzyme in a living body (eye), and thus can be administered to a pharmaceutical composition, quasi-drug composition, or health functional food composition for preventing, alleviating, or treating dry eye.

In addition, phenyllactic acid can be effectively produced when culturing a Lactobacillus plantarum strain in a medium supplemented with phenylaniline and/or calcium carbonate.

### BRIEF DESCRIPTION OF THE DRAWINGS

FIG. 1 is a diagram comparing an ability to produce phenyllactic acid of various Lactobacillus strains.

FIG. 2 is a diagram comparing a phenyllactic acid production amount according to a medium composition and an addition of intermediate metabolites (phenylalanine or phenylpyruvic acid).

FIG. 3 is a diagram confirming an expression level of an HAS2 (Hyaluronan synthase 2) gene according to the addition of phenyllactic acid (PLA) or Lactobacillus fermentation. Untreated group: No treatment with stimulus (blue light), —(control group): Only stimulation treated, PLA: Stimulant treatment and PLA addition, Lactobacillus fermentation: Stimulant treatment and Lactobacillus fermentation added.

FIG. 4 is a diagram confirming an expression level of an HAS2 (hyaluronan synthase 2) gene according to the addition of Lactobacillus plantarum APsulloc 331261 and other Lactobacillus fermentation. Untreated group: No treatment with stimulus (blue light), —(control group): Only stimulation treated.

### DETAILED DESCRIPTION OF THE INVENTION

Hereinafter, the present disclosure is described in detail.

One aspect of the present disclosure relates to a composition for moisturizing a skin or promoting hyaluronic acid production, comprising at least one selected from the group consisting of phenyllactic acid; a strain having phenyllactic acid production ability; a lysate thereof; a culture thereof, a fermentation thereof, and an extract of the strain, lysate, culture or fermentation thereof as an active ingredient.

In an exemplary embodiment, the strain may be a Lactobacillus plantarum strain.

In an exemplary embodiment, the strain may be a Lactobacillus plantarum APsulloc 331261 (Accession Number: KCCM11179P).

In an exemplary embodiment, the strain may convert phenylalanine into phenyllactic acid.

In an exemplary embodiment, the composition may comprise phenyllactic acid in an amount of from 0.1 ppm to 1,000 ppm, more specifically, 0.1 ppm or more, 0.2 ppm or more, 0.3 ppm or more, 0.4 ppm or more, 0.5 ppm or more, 0.6 ppm or more, 0.7 ppm or more, 0.8 ppm or more, 0.9 ppm or more, 1 ppm or more, 2 ppm or more, 3 ppm or more, 4 ppm or more, 5 ppm or more, 6 ppm or more, 7 ppm or more, or 8 ppm or more, or 1,000 ppm or less, 900 ppm or less, 800 ppm or less, 700 ppm or less, 600 ppm or less, 500 ppm or less, 400 ppm or less, 300 ppm or less, 200 ppm or less, 100 ppm or less, 90 ppm or less, 80 ppm or less, 70 ppm or less, 60 ppm or less, 50 ppm or less, 40 ppm or less, 30 ppm or less, 20 ppm or less, 19 ppm or less, 18 ppm or less, 17 ppm or less, 16 ppm or less, 15 ppm or less, 14 ppm or less, 13 ppm or less, 12 ppm or less, 11 ppm or less, or 10 ppm or less, but is not limited thereto. Within the above range, the expression of hyaluronic acid producing enzyme in skin cells may be effectively increased without causing cytotoxicity, skin irritation, etc., thereby protecting the skin from dryness caused by blue light, ultraviolet rays, etc.

In an exemplary embodiment, the phenyllactic acid may be administered at a dosage of 0.1 to 100 mg/kg/day. If the above phenyllactic acid dosage is less than 0.1 mg/kg/day, the skin moisturizing effect or hyaluronic acid production promotion effect may be minimal, and if the above phenyllactic acid dosage exceeds 100 mg/kg/day, cytotoxicity may be exhibited or the skin moisturizing effect or hyaluronic acid production promotion effect efficiency may be reduced. More specifically, the daily dosage of phenyllactic acid may be 0.1 mg/kg/day or more, 0.2 mg/kg/day or more, 0.3 mg/kg/day or more, 0.4 mg/kg/day or more, 0.5 mg/kg/day or more, 0.6 mg/kg/day or more, 0.7 mg/kg/day or more, 0.8 mg/kg/day or more, 0.9 mg/kg/day or more, 1.0 mg/kg/day or more, 2.0 mg/kg/day or more, 3.0 mg/kg/day or more, 4.0 mg/kg/day or more, 5.0 mg/kg/day or more, 6.0 mg/kg/day or more, 7.0 mg/kg/day or more, 8.0 mg/kg/day or more, 9.0 mg/kg/day or more, or 10.0 mg/kg/day or more, 100 mg/kg/day or less, 95 mg/kg/day or less, 90 mg/kg/day or less, 85 mg/kg/day or less, 80 mg/kg/day or less, 75 mg/kg/day or less, 70 mg/kg/day or less, 65 mg/kg/day or less, 60 mg/kg/day or less, 55 mg/kg/day or less, 50 mg/kg/day or less, 45 mg/kg/day or less, 40 mg/kg/day or less, 35 mg/kg/day or less, 30 mg/kg/day or less, 25 mg/kg/day or less, 20 mg/kg/day or less, 15 mg/kg/day or less, or 10 mg/kg/day or less, but is not limited thereto. The administration may be conducted once a day or in several divided doses. For example, it may be administered 2 to 24 times a day, 1 to 2 times every 3 days, 1 to 6 times a week, 1 to 10 times every 2 weeks, 1 to 15 times every 3 weeks, 1 to 3 times every 4 weeks, or 1 to 12 times a year, but is not limited thereto.

In an exemplary embodiment, at least one selected from the group consisting of the strain having the phenyllactic acid production ability; a lysate thereof, a culture thereof, a fermentation thereof, and an extract of the strain, lysate, culture or fermentation thereof may be administered in the effective amount so that a daily dosage of the phenyllactic acid is 0.1 to 100 mg/kg/day.

In an exemplary embodiment, when the active ingredient is a fermentation of the strain having the ability to produce phenyllactic acid, the active ingredient may be administered in an amount of 0.25 to 250 g/kg/day. More specifically, the dosage of the active ingredient may be 0.25 g/kg/day or more, 0.5 g/kg/day or more, 0.75 g/kg/day or more, 1.0 g/kg/day or more, 1.25 g/kg/day or more, 1.5 g/kg/day or more, 1.75 g/kg/day or more, 2.0 g/kg/day or more, 2.25 g/kg/day or more, 2.5 g/kg/day or more, 2.75 g/kg/day or more, 3.0 g/kg/day or more, 3.25 g/kg/day or more, 3.5 g/kg/day or more, 3.75 g/kg/day or more, 4.0 g/kg/day or more, 4.25 g/kg/day or more, 4.5 g/kg/day or more, 4.75 g/kg/day or more, or 5.0 g/kg/day or more, or 250 g/kg/day or less, 200 g/kg/day or less, 150 g/kg/day or less, 100 g/kg/day or less, 90 g/kg/day or less, 80 g/kg/day or less, 70 g/kg/day or less, 60 g/kg/day or less, 50 g/kg/day or less, 40 g/kg/day or less, 30 g/kg/day or less, 20 g/kg/day or less, or 10 g/kg/day or less, but is not limited thereto.

The term “skin” herein includes mucous membranes. An example of mucous membranes is the eye. Dry eye refers to a condition in which the surface of the eye becomes dry due to insufficient production or secretion of tears or excessive evaporation, which may cause symptoms such as discomfort, congestion, foreign body sensation, and numbness.

The term “administration” here means providing a composition according to the present disclosure to a subject of administration by any appropriate method, and refers to a broad range including application, absorption, and ingestion. In this case, the subject of administration refers to all animals such as humans, monkeys, dogs, goats, pigs, or rats to which the composition may be administered.

The term “prevention” herein means any act of suppressing or delaying dry eye by administering the pharmaceutical composition. The term “treatment” refers to any act of relieving or beneficially changing the symptoms of a subject suspected of or developing dry eye by administering the pharmaceutical composition.

The term “alleviation” herein means any act of relieving or beneficially changing dry eye by administering the composition.

In an exemplary embodiment, the composition may be administered to a subject in need of increased expression of hyaluronic acid producing enzymes in skin cells or within the eye.

In an exemplary embodiment, the composition may be a transdermal composition or an oral composition.

In an exemplary embodiment, the composition may be at least one selected from the group consisting of a skin moisturizing cosmetic composition, a quasi-drug composition, a general food composition, a health functional food composition, and a non-therapeutic oral composition.

In an exemplary embodiment, the composition may be at least one selected from the group consisting of a pharmaceutical composition, quasi-drug composition, health functional food composition, and therapeutic or non-therapeutic oral composition for preventing, alleviating, or treating dry eye.

In an exemplary embodiment, the composition may comprise 0.001 to 99.9 wt % of the active ingredient. For example, it may contain 0.01 to 20.0 wt % or 0.1 to 10.0 wt %, but is not limited thereto.

In an exemplary embodiment, the cosmetic composition may be prepared in a typical emulsified or solubilized formulation. The emulsified formulation may include a nutritional toner, cream, essence, etc., and the solubilized formulation may include a softening toner, etc. An appropriate formulation may be, but is not limited to, a solution, gel, solid or paste-like anhydrous products, emulsions obtained by dispersing oil phase in water phase, suspensions, microemulsions, microcapsules, micro granulocytes or ion type (liposomes), bionic vesicular dispersants, cream, toner, lotion, powder, ointment, spray, or concealed stick. In addition, the cosmetic composition may be in a form of foam or aerosol composition comprising more of compressed propellant.

In an exemplary embodiment, the cosmetic composition may additionally comprise commonly used auxiliary agents such as fatty substances, organic solvents, solubilizers, thickening agents and gelling agents, softeners, antioxidants, suspending agents, stabilizers, foaming agents, fragrances, surfactants, water, ionic or non-ionic emulsifiers, fillers, sequestering agents, chelating agents, preservatives, vitamins, blocking agents, wetting agents, essential oils, dyes, pigments, hydrophilic or lipophilic activators, lipid vesicles, or any other ingredients commonly used in a cosmetic composition.

In an exemplary embodiment, when the composition is used as an additive for general food or health functional food, it may be added as is or used together with other food or food ingredients, and may be used appropriately according to a conventional method. The amount of active ingredients mixed may be appropriately determined according to each purpose of use, such as prevention, health, or treatment. The food formulation may be in the form of powder, granule, pill, tablet, capsule, or any form of general food or beverage.

In an exemplary embodiment, there is no particular limitation on the type of the general food or health functional food, and examples of foods to which the composition may be added include dairy products including meat, confectionery, noodles, gum, ice cream, various soups, beverages, tea, drinks, alcoholic beverages, and vitamin complexes, and all foods in the conventional sense may be included.

In an exemplary embodiment, the beverage among the general foods or health functional foods may contain various flavoring agents or natural carbohydrates as additional ingredients, like a regular beverage. The natural carbohydrates may be monosaccharides, such as glucose and fructose, disaccharides, such as maltose and sucrose, polysaccharides, such as dextrin and cyclodextrin, and sugar alcohols, such as xylitol, sorbitol, and erythritol. As the sweetening agents, there may be used, for example, natural sweetening agents, such as thaumatin and stevia extract, and synthetic sweetening agents, saccharine and aspartame. The total weight of the natural carbohydrates may be from about 0.01 to about 0.04 g, from about 0.02 to about 0.03 g, based on 100 mL of the beverage according to the present disclosure, but is not limited thereto.

In an exemplary embodiment, in addition to the above, the general foods or health functional foods of the present disclosure may contain various nutrients, vitamins, electrolytes, flavoring agents, coloring agents, pectic acid and its salts, alginic acid and its salts, organic acids, protective colloid thickeners, pH adjusting agents, stabilizers, preservatives, glycerin, alcohol, and carbonating agents used in carbonated beverages. The foods of the present disclosure may further contain flesh for the production of natural fruit juices, fruit juice beverages, and vegetable beverages. Such ingredients may be used independently or as a mixture thereof. The proportions of such additives are not limited but are typically selected from the range of 0.01 to 0.1 parts by weight, based on 100 parts by weight of the health functional foods of the present disclosure.

In an exemplary embodiment, the pharmaceutical composition may be provided in all dosage forms suitable for topical application. For example, the composition may be administered by oral, transdermal, intravenous, intramuscular, subcutaneous injection. As an example, the pharmaceutical composition may be an injection, a skin external preparation, a suspension, an emulsion, a gel, a patch, or a spray, but is not limited thereto. The dosage form may be easily prepared according to a conventional method in the art, and a surfactant, an excipient, a wettable powder, an emulsification promoter, a suspending agent, a salt or buffer for controlling osmotic pressure, a colorant, a fragrance, a stabilizer, a preservative or other commonly used adjuvants may be suitably used.

In an exemplary embodiment, the active ingredients of the pharmaceutical composition vary according to the age, sex, body weight, pathological conditions and severity of the subject, the administration route or the judgment of the prescriber. Determining the appropriate dosage based on these factors is within the skill of a person skilled in the art.

In an exemplary embodiment, when the composition is used as a quasi-drug additive, the composition may be added as it is or used together with other quasi-drugs or quasi-drug ingredients, and may be used appropriately according to a conventional method. The amount of the active ingredient mixed may be suitably determined depending on the intended use. The ingredients included in the quasi-drug composition may include, in addition to the above-mentioned active ingredient, ingredients commonly used in quasi-drug compositions, and may include, for example, an abrasive, a wetting agent, a binder, a foaming agent, a sweetener, a preservative, a pharmaceutical ingredient, a flavoring agent, a pigment, a solvent, a whitening agent, a solubilizer, or a pH adjusting agent.

Another aspect of the present disclosure relates to a method for producing phenyllactic acid comprising culturing a Lactobacillus plantarum strain.

In an exemplary embodiment, the strain may be a Lactobacillus plantarum APsulloc 331261 (Accession Number: KCCM11179P) strain.

In an exemplary embodiment, the culturing may comprise the Lactobacillus plantarum strain in a medium supplemented with phenylalanine and/or phenylpyruvic acid.

In an exemplary embodiment, the culturing may comprise the Lactobacillus plantarum APsulloc 331261 (Accession Number: KCCM11179P) strain in a medium supplemented with phenylalanine.

In an exemplary embodiment, the medium may comprise phenylalanine in an amount of 1 mM or more and 150 mM or less, more specifically, 1 mM or more, 5 mM or more, 10 mM or more, 15 mM or more, 20 mM or more, 25 mM or more, 30 mM or more, 35 mM or more, 40 mM or more, 45 mM or more, 50 mM or more, 55 mM or more, 60 mM or more, 65 mM or more, 70 mM or more, 75 mM or more, or 80 mM or more, or 150 mM or less, 145 mM or less, 140 mM or less, 135 mM or less, 130 mM or less, 125 mM or less, 120 mM or less, 115 mM or less, 110 mM or less, 105 mM or less, 100 mM or less, or 95 mM or less, but is not limited thereto. If the strain is cultured in a medium to which 1 mM or more of phenylalanine is added, phenyllactic acid may be effectively produced, and phenylalanine exceeding 150 mM in the medium may have a negative effect on the growth of the strain, and the efficiency of phenyllactic acid production may be reduced.

In an exemplary embodiment, the medium may be a medium to which calcium carbonate is added for culturing the strain. The pH of the medium may be adjusted by adding calcium carbonate, thereby increasing the production of phenyllactic acid.

In an exemplary embodiment, the medium may have a pH of 7.0 or greater, 7.1 or greater, 7.2 or greater, 7.3 or greater, 7.5 or greater, 7.6 or greater, 7.7 or greater, 7.8 or greater, 7.9 or greater, or 8.0 or greater, but is not limited thereto.

In an exemplary embodiment, the culturing may comprise the Lactobacillus plantarum strain in a medium supplemented with phenylalanine and/or phenylpyruvic acid; and calcium carbonate.

In an exemplary embodiment, the culturing may comprise the Lactobacillus plantarum APsulloc 331261 (Accession Number: KCCM11179P) strain in a medium supplemented with phenylalanine and calcium carbonate.

Another aspect of the present disclosure relates to a method for moisturizing a skin, comprising administering to a subject an effective amount of a composition comprising at least one selected from the group consisting of phenyllactic acid; a strain having phenyllactic acid production ability; a lysate thereof; a culture thereof, a fermentation thereof, and an extract of the strain, lysate, culture or fermentation thereof.

Another aspect of the present disclosure relates to a method for promoting hyaluronic acid production, comprising administering to a subject an effective amount of a composition comprising at least one selected from the group consisting of phenyllactic acid; a strain having phenyllactic acid production ability; a lysate thereof, a culture thereof, a fermentation thereof, and an extract of the strain, lysate, culture or fermentation thereof.

Another aspect of the present disclosure relates to a method for preventing, alleviating, or treating dry eye, comprising administering to a subject an effective amount of a composition comprising at least one selected from the group consisting of phenyllactic acid; a strain having phenyllactic acid production ability; a lysate thereof, a culture thereof, a fermentation thereof, and an extract of the strain, lysate, culture or fermentation thereof.

Another aspect of the present disclosure relates to a use of at least one selected from the group consisting of phenyllactic acid; a strain having phenyllactic acid production ability; a lysate thereof, a culture thereof, a fermentation thereof, and an extract of the strain, lysate, culture or fermentation thereof, for the manufacture of a composition for moisturizing the skin; a composition for promoting hyaluronic acid production; or a composition for preventing, alleviating or treating dry eye.

Another aspect of the present disclosure relates to at least one selected from the group consisting of phenyllactic acid; a strain having phenyllactic acid production ability; a lysate thereof; a culture thereof, a fermentation thereof, and an extract of the strain, lysate, culture or fermentation thereof, for moisturizing the skin; promoting hyaluronic acid production; or preventing, alleviating or treating dry eye.

Another aspect of the present disclosure relates to a non-therapeutic use of at least one selected from the group consisting of phenyllactic acid; a strain having phenyllactic acid production ability; a lysate thereof; a culture thereof, a fermentation thereof, and an extract of the strain, lysate, culture or fermentation thereof, for moisturizing the skin; promoting hyaluronic acid production; or preventing or alleviating dry eye.

Hereinafter, the configuration and effects of the present disclosure will be described more specifically with reference to Examples. However, Examples below are provided only for the purpose of illustration to help understanding of the present disclosure, and the scope and range of the present disclosure are not limited thereby.

### [Experimental Example 1] Isolation of Phenyllactic Acid from Various Strains

In the Experimental Example, phenyllactic acid was isolated from various strains of the genus. Phenyllactic acid was isolated from various strains of the genus, including Lactobacillus strains of the genus Lactobacillus, including Lactobacillus acidophilus KCTC3164, Lactobacillus casei KCTC3109, Lactobacillus plantarum KCTC3108, Lactobacillus sakei KCTC3603, and Lactobacillus plantarum APsulloc 331261 (KCCM11179P) strain. The strains were inoculated into MRS broth and static-cultured at 35° C. for 3 days. The culture was centrifuged at 1,800×g for 20 minutes at 4° C. to separate the culture medium and the cells. The separated culture medium was filtered through a 0.2 μm filter to completely remove the cells. The culture medium prepared as above was compared for the production of phenyllactic acid through HPLC analysis.

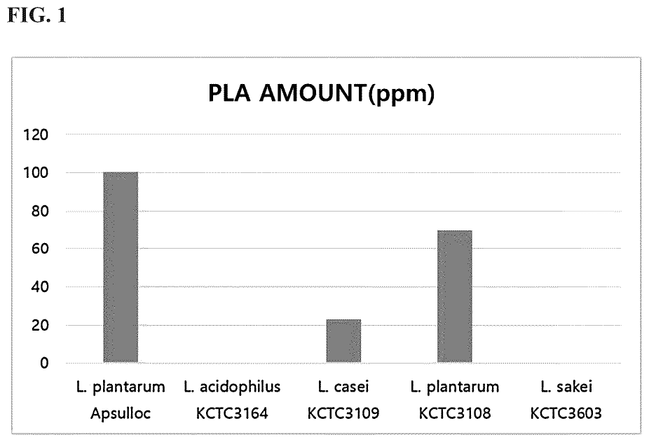

As a result, as shown in Table 1 and FIG. 1 below, the phenyllactic acid production ability of Lactobacillus plantarum strains was superior to that of other strains. In particular, the phenyllactic acid production of the Lactobacillus plantarum APsulloc 331261 (KCCM11179P) strain was high at about 100 ppm, and the phenyllactic acid production of the Lactobacillus plantarum KCTC3108 strain was about 70.0 ppm, whereas the phenyllactic acid production of the Lactobacillus acidophilus KCTC3164, Lactobacillus casei KCTC3109, and Lactobacillus sakei KCTC3603 strains were undetectable, 22.8 ppm, and undetectable, respectively. Therefore, it was confirmed that among various strains of the genus Lactobacillus plantarum, especially Lactobacillus plantarum APsulloc 331261 (KCCM11179P) strain has excellent phenyllactic acid production ability. Accordingly, Lactobacillus plantarum APsulloc 331261 (KCCM11179P) strain was used in Experimental Example described below.

TABLE 1
Comparison of phenyllactic acid production ability of various strains

| | | | | | PLA |
|---|---|---|---|---|---|
| | | | | Description | amount |
| No. | Genus | Species | Strain | of strain | (ppm) |
| Example 1 | Lactobacillus | plantarum | APsulloc | Green tea-derived | 100.23 |
| | | | 331261 | lactic acid | |
| | | | | bacteria | |
| Example 2 | Lactobacillus | plantarum | KCTC3108 | type strain, | 69.96 |
| | | | | Pickled cabbage | |
| | | | | derived | |
| Example 3 | Lactobacillus | plantarum | KCTC13093 | Kimchi lactic | 82.85 |
| | | | | acid bacteria | |
| Example 4 | Lactobacillus | plantarum | KCTC13093 | Kimchi lactic | 82.85 |
| | | | | acid bacteria | |
| Example 5 | Lactobacillus | plantarum | 299v | CENOVIS | 88.52 |
| | | | | Superbiotics | |
| | | | | Probiotics | |
| Comparative | Lactobacillus | rhamnosus | GG (LGG) | Denps | 20.24 |
| Example 1 | | | | Denmark | |
| | | | | Probiotics Story | |
| Comparative | Lactobacillus | acidophilus | KCTC3164 | type strain, | N.D. |
| Example 2 | | | | PURCHASED | |
| | | | | FROM KCTC | |
| Comparative | Lactobacillus | casei | KCTC3109 | type strain, | 22.75 |
| Example 3 | | | | PURCHASED | |
| | | | | FROM KCTC | |
| Comparative | Lactobacillus | sakei | KCTC3603 | type strain, | N.D. |
| Example 4 | | | | PURCHASED | |
| | | | | FROM KCTC | |
| Comparative | Lactobacillus | brevis | Iwha KSD | Isolated directly | N.D. |
| Example 5 | | | | | |
| Comparative | Lactobacillus | sakei | MD_{—} | Isolated directly | N.D. |
| Example 6 | | | honeysucle | | |
| Comparative | Lactobacillus | jensenii | KCTC5194 | PURCHASED | N.D. |
| Example 7 | | | | FROM KCTC | |
| Comparative | Lactococcus | lactis ssp lactis | BJL-L1 | Isolated directly | N.D. |
| Example 8 | | | | | |
| Comparative | Lactococcus | garvieae | BR-L1 | Isolated directly | N.D. |
| Example 9 | | | | | |
| Comparative | Lactococcus | garvieae | Narciuss-L1 | Isolated directly | N.D. |
| Example 10 | | | | | |
| Comparative | Lactococcus | garvieae | Narciuss-L2 | Isolated directly | N.D. |
| Example 11 | | | | | |
| Comparative | Lactococcus | lactis | W-Camellia- | Isolated directly | N.D. |
| Example 12 | | | L2 | | |
| Comparative | Lactococcus | lactis | R_Camellia_{—} | Isolated directly | N.D. |
| Example 13 | | | L1 | | |
| Comparative | Lactococcus | lactis ssp lactis | KCTC3769 | PURCHASED | N.D. |
| Example 14 | | | | FROM KCTC | |
| Comparative | Lactococcus | lactis ssp lactis | JBF-M173 | Isolated directly | N.D. |
| Example 15 | | | | | |
| Comparative | Lactococcus | lactis ssp lactis | J-crater-L1 | Isolated directly | N.D. |
| Example 16 | | | | | |
| Comparative | Lactococcus | lactis ssp lactis | J-crater-L2 | Isolated directly | N.D. |
| Example 17 | | | | | |
| Comparative | Lactococcus | lactis ssp lactis | J-crater-L3 | Isolated directly | N.D. |
| Example 18 | | | | | |
| Comparative | Lactococcus | lactis ssp lactis | J-basidio-L1 | Isolated directly | N.D. |
| Example 20 | | | | | |
| Comparative | Lactococcus | lactis ssp lactis | J-basidio-L2 | Isolated directly | N.D. |
| Example 21 | | | | | |
| Comparative | Lactococcus | lactis ssp lactis | J-basidio-L3 | Isolated directly | N.D. |
| Example 22 | | | | | |
| Comparative | Leuconostoc | citreum | SRN-L1 | Isolated directly | 31.11 |
| Example 23 | | | | | |
| Comparative | Leuconostoc | pseudomesenteroides | GTF-L1 | Isolated directly | 17.39 |
| Example 24 | | | | | |
| Comparative | Leuconostoc | pseudomesenteroides | GTF-L2 | Isolated directly | 18.45 |
| Example 25 | | | | | |
| Comparative | Leuconostoc | pseudomesenteroides | GTF-L3 | Isolated directly | 32.47 |
| Example 26 | | | | | |
| Comparative | Leuconostoc | citreum | BL-L5 | Isolated directly | 22.70 |
| Example 27 | | | | | |
| Comparative | Leuconostoc | mesenteroides | R-Camellia- | Isolated directly | N.D. |
| Example 28 | | | L2 | | |
| Comparative | Leuconostoc | mesenteroides | R-Camellia- | Isolated directly | 10.75 |
| Example 29 | | | L3 | | |
| Comparative | Leuconostoc | mesenteroides | R-Camellia- | Isolated directly | 8.35 |
| Example 30 | | | L4 | | |
| Comparative | Leuconostoc | mesenteroides | R-Camellia- | Isolated directly | 11.60 |
| Example 31 | | | L5 | | |
| Comparative | Bifidobacterium | longum | KCTC3128 | type strain, | N.D. |
| Example 32 | | | | PURCHASED | |
| | | | | FROM KCTC | |
| Comparative | Bifidobacterium | lactis | KCTC5854 | type strain, | N.D. |
| Example 33 | | | | PURCHASED | |
| | | | | FROM KCTC | |
| Comparative | Enterococcus | faecalis | BF-L1 | Isolated directly | N.D. |
| Example 34 | | | | | |
| Comparative | Enterococcus | faecalis | BL-L1 | Isolated directly | N.D. |
| Example 35 | | | | | |
| Comparative | Enterococcus | faecalis | BL-L2 | Isolated directly | N.D. |
| Example 36 | | | | | |
| Comparative | Enterococcus | faecalis | BL-L3 | Isolated directly | N.D. |
| Example 37 | | | | | |
| Comparative | Enterococcus | faecium | nuruk-1 | Isolated directly | N.D. |
| Example 38 | | | | | |
| Comparative | Enterococcus | hirae | nuruk-2 | Isolated directly | N.D. |
| Example 39 | | | | | |
| Comparative | Pediococcus | pentosaceus | lotus-f-L1 | Isolated directly | N.D. |
| Example 40 | | | | | |
| Comparative | Pediococcus | pentosaceus | lotus-f-L2 | Isolated directly | N.D. |
| Example 41 | | | | | |
| Comparative | Pediococcus | pentosaceus | lotus-f-L3 | Isolated directly | N.D. |
| Example 42 | | | | | |
| Comparative | Streptococcus | thermophilus | KCTC3658 | type strain, | N.D. |
| Example 43 | | | | PURCHASED | |
| | | | | FROM KCTC | |
| Comparative | Weissella | cibaria | JBN-L1 | Isolated directly | N.D. |
| Example 44 | | | | | |
| Comparative | Weissella | cibaria | JBN-L2 | Isolated directly | N.D. |
| Example 45 | | | | | |
| Comparative | Weissella | cibaria | BL-L4 | Isolated directly | N.D. |
| Example 46 | | | | | |
| Comparative | Weissella | cibaria | KCTC3807 | type strain, | N.D. |
| Example 47 | | | | PURCHASED | |
| | | | | FROM KCTC | |
| Comparative | Weissella | cibaria | KCTC3817 | type strain, | N.D. |
| Example 48 | | | | PURCHASED | |
| | | | | FROM KCTC | |

### [Experimental Example 2] Confirmation of Increased Phenyllactic Acid Production by Lactobacillus Strains According to the Composition of the Medium and the Addition of Intermediate Metabolites

In Experimental Example above, the Lactobacillus plantarum APsulloc 331261 (KCCM11179P) strain was inoculated into a medium containing MRS broth, phenylalanine and phenylpyruvic acid, and the production of phenyllactic acid was confirmed. Specifically, 20, 40, and 80 mM of phenylalanine or phenylpyruvic acid were added to MRS broth, or 1% concentration of calcium carbonate was added to 40 mM phenylalanine or phenylpyruvic acid, and the Lactobacillus strain was inoculated in the same manner, followed by static culture at 35° C. for 3 days, and the production of phenyllactic acid was confirmed. The culture was centrifuged at 1,800×g for 20 minutes at 4° C. in the same manner as in Example above to separate the culture medium and the cells. The separated culture medium was filtered through a 0.2 μm filter to completely remove the cells. The culture medium prepared as above was compared for the production of phenyllactic acid through HPLC analysis.

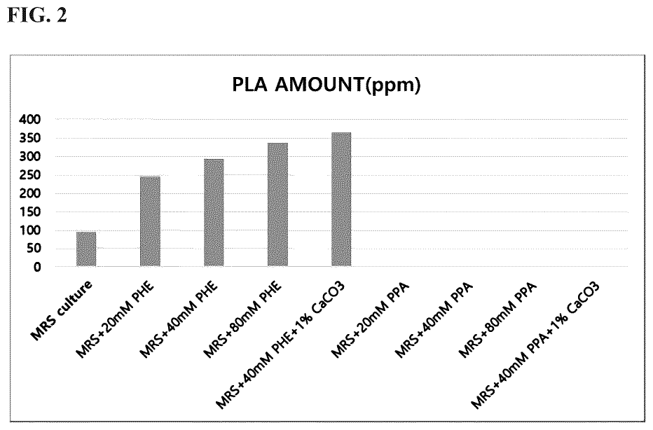

As a result, as shown in FIG. 2, the production of phenyllactic acid increased depending on the administered concentration of phenylalanine, and the highest production was 363.5 ppm when the pH was adjusted with calcium carbonate. On the other hand, when phenylpyruvic acid was added, the Lactobacillus plantarum APsulloc 331261 (KCCM11179P) strain did not grow and did not produce phenyllactic acid.

### [Experimental Example 3] Isolation of Phenyllactic Acid from Lactobacillus Culture Medium

The Lactobacillus plantarum APsulloc 331261 (KCCM11179P) strain used in Experimental Example above was inoculated into a medium containing MRS broth, phenylalanine at a concentration of 40 mM, and calcium carbonate at a concentration of 1%, and the culture medium was cultured for 3 days. The cells were removed by centrifugation, and 1 liter was prepared. An equal amount of ethyl acetate was added to the prepared culture medium and the reaction was performed, and the water layer and the ethyl acetate layer of the culture medium were waited for separation. The separated ethyl acetate layer was heated and distilled, and an appropriate amount of water (about 100 mL) was added to the remaining concentrate to prepare a Lactobacillus culture extract containing phenyllactic acid. It was confirmed that the Lactobacillus culture extract contained about 3,000 ppm of phenyllactic acid. [Experimental Example 4] Confirmation of the effect of phenyllactic acid on promoting hyaluronic acid production Human keratinocytes (HaCaT) were cultured in 35 mm culture dishes at 37° C. in a 5% CO₂ incubator. After 24 hours, PLA 50 M (8.3 ppm) and the culture extract of Experimental Example 3 were treated in DMEM (w/gentamicin/amphotericin B, w/o FBS. Lonza) medium. 0.5%, 0.25%, and 0.125% of the culture extract of Lactobacillus plantarum APsulloc 331261 (KCCM11179P) strain containing approximately 3000 ppm of PLA were added and pretreated for 24 hours. After pretreatment for 24 hours, 1.25 ml of HBSS (Lonza) was added and irradiated with blue light 410 nm 120 J/cm², 50 M PLA, 0.5%, 0.25%, 0.125% of Lactobacillus culture extract containing PLA were added to DMEM (w/gentamicin/amphotericin B, w/o FBS. Lonza) medium and cultured for 24 hours at 37° C. in a 5% CO₂ incubator. After collecting the cells, RNA was isolated and cDNA was synthesized through RT-PCR (reverse transcriptional polymerase chain reaction). The synthesized cDNA was used to measure the gene expression amount of HAS2 (Hyaluronan synthase 2) by performing Taqman real-time PCR.

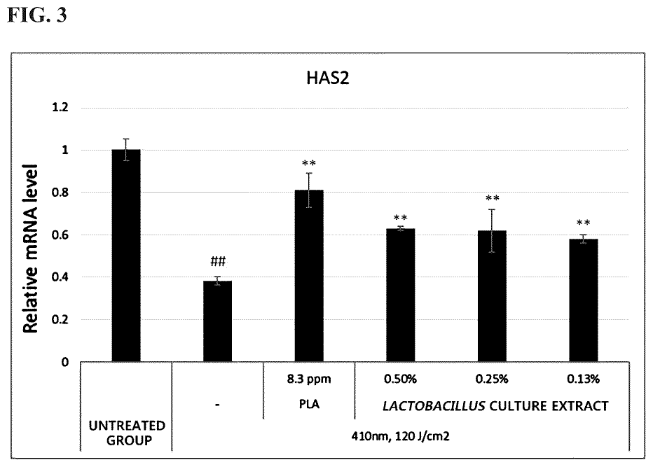

As a result, as shown in FIG. 3, it was confirmed that not only 50 M PLA but also 0.5%, 0.25%, and 0.125% Lactobacillus culture extracts containing PLA significantly improved the decrease in HAS2 gene expression caused by blue light.

In addition, the hyaluronic acid production promotion effect of the culture medium of Lactobacillus strains prepared as shown in Table 1 was compared. The strains Lactobacillus plantarum APsulloc 331261 (KCCM11179P), Lactobacillus plantarum KCTC3108, Lactobacillus acidophilus KCTC3164, and Lactobacillus sakei KTCT3603 were inoculated into MRS broth, and 0.5% of the culture medium static-cultured at 35° C. for 3 days was added, and the gene expression amount of HAS2 (Hyaluronan synthase 2) was measured in the same manner as above.

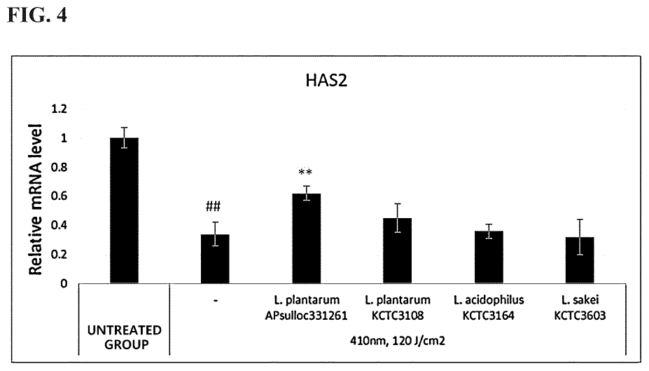

As a result, as shown in FIG. 4, the Lactobacillus plantarum strain culture medium significantly improved the decrease in HAS2 gene expression caused by blue light compared to other Lactobacillus strain culture medium, and in particular, it was confirmed that the Lactobacillus plantarum APsulloc 331261 (KCCM11179P) strain culture medium effectively improved the decrease in HAS2 gene expression.

### [Deposition Information]

Depositary Authority: Korea Center for Microorganism Conservation (KCCM)
Accession Number: KCCM11179P
Date of deposit: 20110328

We claim:

1. A method for moisturizing a skin or promoting hyaluronic acid production, comprising administering to a subject an effective amount of a composition comprising at least one selected from the group consisting of phenyllactic acid; a strain having phenyllactic acid production ability; a lysate thereof; a culture thereof; a fermentation thereof; and an extract of the strain, lysate, culture or fermentation thereof as an active ingredient.

2. The method of claim 1, wherein the strain is a Lactobacillus plantarum strain.

3. The method of claim 1, wherein the strain is a Lactobacillus plantarum APsulloc 331261 (Accession Number: KCCM11179P).

4. The method of claim 1, wherein the strain converts phenylalanine and/or phenylpyruvic acid into phenyllactic acid.

5. The method of claim 1, wherein the composition comprises the phenyllactic acid in an amount of 0.1 ppm or more and 1,000 ppm or less.

6. The method of claim 1, wherein a daily dosage of the phenyllactic acid is 0.1 to 100 mg/kg/day.

7. The method of claim 1, wherein the at least one selected from the group consisting of the strain having the phenyllactic acid production ability; a lysate thereof; a culture thereof; a fermentation thereof; and an extract of the strain, lysate, culture or fermentation thereof is administered in the effective amount so that a daily dosage of the phenyllactic acid is 0.1 to 100 mg/kg/day.

8. The method of claim 1, wherein when the active ingredient is the fermentation of the strain having the phenyllactic acid production ability, the active ingredient is administered at a dosage of 0.25 to 250 g/kg/day.

9. The method of claim 1, wherein the composition is administered to the subject in need of increased expression of a hyaluronic acid producing enzyme in a skin cell.

10. The method of claim 1, wherein the composition is a transdermal composition or an oral composition.

11. The method of claim 1, wherein the composition is a cosmetic composition, a quasi-drug composition, a general food composition, or a health functional food composition.

12. A method for producing phenyllactic acid comprising culturing a Lactobacillus plantarum strain.

13. The method of claim 12, wherein the strain is a Lactobacillus plantarum APsulloc 331261 (Accession Number: KCCM11179P) strain.

14. The method of claim 12, wherein the culturing comprises the Lactobacillus plantarum strain in a medium supplemented with phenylalanine and/or phenylpyruvic acid; and/or calcium carbonate.

15. The method of claim 12, wherein the culturing comprises the Lactobacillus plantarum APsulloc 331261 (Accession Number: KCCM11179P) strain in a medium supplemented with phenylalanine and/or calcium carbonate.
